(19) United States
(12) Patent Application Publication
(10) Pub. No.: US 2012/0093809 A1
(43) Pub. Date: Apr. 19, 2012

(54) USE OF ALKANOYL L-CARNITINE IN COMBINATION WITH CHEMOTHERAPEUTIC AGENTS FOR THE TREATMENT OF NEOPLASMS

(75) Inventors: Claudio Cavazza, Rome (IT); Giulia Tagliafico, Rome (IT); Paolo Carminati, Milan (IT); Gabriella Singrossi, Milan (IT); Silvia Carminati, Milan (IT); Giuseppe Paolo Carminati, Milan (IT); Claudio Pisano, Aprilia (LT) (IT)

(73) Assignee: SIGMA-TAU INDUSTRIE FARMACEUTICHE RIUNITE S.P.A., Rome (IT)

(21) Appl. No.: 13/256,531

(22) PCT Filed: Dec. 1, 2009
(86) PCT No.: PCT/EP09/66113

§ 371 Date: Dec. 15, 2011

Related U.S. Application Data
(60) Provisional application No. 61/248,543, filed on Oct. 5, 2009.

(30) Foreign Application Priority Data
Dec. 1, 2008 (EP) 08170331.6

(51) Int. Cl. A61K 31/205; A61K 31/282; A61P 35/02; A61K 31/704; A61P 35/00; A61K 39/395; A61K 33/24

(52) U.S. Cl. 424/133.1; 514/556; 514/492; 424/649; 514/34

(57) ABSTRACT

The present invention relates to the use of an alkanoyl L-carnitine selected from the group consisting of acetyl, propionyl, valeryl, isovaleryl and butirryl L-carnitine; in combination with one or more chemotherapeutic agent selected from the group consisting of: a camptothecin derivative; an alkylating agent; an anti-neoplastic anti-metabolite; a platin compound; a topoisomerase inhibitor; a VEGF inhibitor; a tyrosine kinase inhibitor; an EGFR kinase inhibitor; an mTOR kinase inhibitor; an insulin-like growth factor I inhibitor; a Raf kinase inhibitor; a monoclonal antibody; a proteasome inhibitor; a HDAC inhibitor; toxins; and imides; for the treatment of neoplasms.

### FIELD OF THE INVENTION

The invention relates to a method of preventing or treating proliferative diseases or diseases that may be associated with or triggered by persistent angiogenesis in a mammal, particularly a human, with a combination of pharmaceutical agents which comprises: (a) an alkanoyl L-carnitine derivative; and (b) one or more chemotherapeutic agents; in which the dose of acetyl L-carnitine to be administered (to adult human) is higher than 0.5 g/day, preferably higher than 0.8 g/day; most preferably higher than 1 g/day.

Therapeutic effects of combinations of chemotherapeutic agents with an alkanoyl L-carnitine derivative result in lower safe dosages ranges of the chemotherapeutic agent in the combination.

### BACKGROUND OF THE INVENTION

Cancer is a class of diseases in which a group of cells display uncontrolled growth, invasion, and sometimes metastasis.

These three malignant properties of cancers differentiate them from benign cancers, which are self-limited, do not invade or metastasize.

Cancer may affect people at all ages, even foetuses, but the risk for most varieties increases with age. Cancer causes about 13% of all deaths. According to the American Cancer Society, 7.6 million people died from cancer in the world during 2007.

Most cancers can be treated and some cured, depending on the specific type, location, and stage. Once diagnosed, cancer is usually treated with a combination of surgery, chemotherapy and radiotherapy. As research develops, treatments are becoming more specific for different varieties of cancer.

The effectiveness of chemotherapy is often limited by toxicity to other tissues in the body. Radiation can also cause damage to normal tissue.

In the medical field, for treating cancer are widely used combinations of different chemotherapeutic agents. In fact most of the therapeutical protocols provide for the combined use of different antineoplastic agents; this procedure allows to enhance the treatment efficacy because the individual feedback to the agents can change according to the agent adopted.

The use of alkanoyl L-carnitines in the medical field is already known and their preparation process is described in U.S. Pat. No. 4,254,053.

In WO/2000/06134 the use of L-carnitine and its alkanoyl derivatives in the preparation of medicaments with anticancer activity is described. In particular in WO/2000/06134 the following data are reported:
Animals treated with vehicle alone and those treated with paclitaxel (taxol) in combination with acetyl L-carnitine: a statistically significant reduction of the tumour mass was found in the latter (see page 48, lines 16-19);
By contrast, comparison of the group treated with vehicle alone and the one treated with vehicle in combination with acetyl L-carnitine revealed no statistically significant differences in tumour mass growth at any of the observation times (page 48, lines 20-23);
Analysis of the data relating to the comparison between the group treated with paclitaxel (taxol) and the one treated with paclitaxel in combination with acetyl L-carnitine showed no significant differences in tumour weight (page 48, lines 23-26 and page 57, lines 1-7);
As regards the analysis of the number of metastases, the data obtained showed a statistically significant reduction in that number in the groups treated with paclitaxel, with paclitaxel in combination with acetyl L-carnitine and with vehicle in combination with acetyl L-carnitine as compared to the group treated with vehicle alone (page 49, lines 1-4);
In particular, the mice treated with paclitaxel or with paclitaxel in combination with acetyl L-carnitine also showed a reduction in the diameter of the metastases compared to the groups treated with vehicle alone or with vehicle in combination with acetyl L-carnitine (page 49, lines 4-8);
On the basis of analysis of the following data, it was therefore concluded that acetyl L-carnitine does not interfere with the anticancer action of paclitaxel in terms of inhibition of the tumour mass (page 49, lines 8-11);
In addition, acetyl L-carnitine (ALC) showed a significant inhibitory effect on the formation of lung metastases (page 49, lines 11-12);
Paclitaxel treatment caused an inhibition of tumour growth (TVI=88%). Treatment with ALC had no effect on tumour growth, which was similar to that in control group tumours. Combined treatment with paclitaxel plus ALC showed an anticancer efficacy (TVI=90%) almost identical to that achieved with paclitaxel alone, confirming that ALC did not interfere with the cytotoxic activity of paclitaxel (page 61 lines 4-9);
In the paclitaxel+propionyl L-carnitine (PLC) group versus the control group, with p<0.003, and only at the last observation time (day 46) did the significance level drop to p<0.034. It should be noted that the values for the paclitaxel group on day 46 were not significantly different from the control group values (page 66 last line and page 67 lines 1-4);
Only the control group was significantly different from the Paclitaxel+PLC group, with p<0.05 (page 67 last two lines).

It is important to note that in WO/2000/06134 ALC was administered orally at a dose of 100 mg/kg/mice. This does would correspond to a dose of about 0.5 g per day for administration to adult humans (see for example “Guidance for Industry and Reviewers; Estimating the Safe Starting Dose in Clinical Trials for Therapeutics in Adult Healthy Volunteers; Division of Drug Information, HFD-240; Center for Drug Evaluation and Research; Food and Drug Administration; 5600 Fishers Lane; Rockville, Md. 20857; http://www.fda.gov/cder/guidance/index.htm”—Table at page 233).

In Clinical Cancer Research Vol. 9; Nov. 15, 2003; p. 5756-5767; it is reported that ALC protects the mice from the lethal toxicity and from the neurotoxicity due to the use of the antitumor drug tested. About the antitumor activity in this publication it is reported that cisplatin alone significantly reduced the number of lung metastases and that the combination of ALC with cisplatin did not influence the antimetastatic or the antitumor effects of cisplatin.

It must be noted that the dose of ALC used in vivo (in mice) was of 100 mg/kg/day p.o. (which in adult human corresponds to about 0.5 g/day) and that the concentration of ALC used in vitro experiments was of 1 mM. It is also to be noted that the dose of cisplatin used in this paper ranges from 6 to 8 mg/kg (see Table 5).

In WO/2004/043454 the use of acetyl L-carnitine for the prevention and/or treatment of peripheral neuropathies induced by anticancer agents is described.

It is well-known that the use of anticancer agents in chemo therapy causes a large number of toxic or side effects which may lead to a reduction of the dose of the agent administered, and occasionally to discontinuation of the therapy itself. The reduction of the dose of the agent administered reduces the therapeutic efficacy of the anticancer agent.

Therefore the discovery of agents useful for increasing the pharmacological activity of anticancer agents remains a perceived need in the medical field.

Tumor protein p53 is a transcription factor that in humans is encoded by the TP53 gene. p53 is important in multicellular organisms, where it regulates the cell cycle and thus functions as a tumor suppressor that is involved in preventing cancer. This effect is observed with p53 from a variety of species, including humans, rodents, frogs, and fish. In a normal cell p53 is inactivated by its negative regulator, mdm2. Upon DNA damage or other stress, various pathways will lead to the dissociation of the p53 and mdm2 complex. Once activated, p53 will either induce a cell cycle arrest to allow repair and survival of the cell or apoptosis to discard the damage cell. How p53 makes this choice is currently unknown. p53 has many anticancer mechanisms, and plays a role in apoptosis, genetic stability, and inhibition of angiogenesis.

Mutant p53 can no longer bind DNA in an effective way, and as a consequence the p21 protein is not made available to act as the ‘stop signal’ for cell division. Thus cells divide uncontrollably, and form tumors. If the TP53 gene is damaged, tumor suppression is severely reduced. People who inherit only one functional copy of the TP53 gene will most likely develop tumors in early adulthood, a disease known as Li-Fraumeni syndrome. The TP53 gene can also be damaged in cells by mutagens (chemicals, radiation, or viruses), increasing the likelihood that the cell will begin decontrolled division. More than 50 percent of human tumors contain a mutation or deletion of the TP53 gene. Increasing the amount of p53, which may initially seem a good way to treat tumors or prevent them from spreading, is in actuality not a usable method of treatment, since it can cause premature aging.

However, restoring endogenous p53 function holds a lot of promise. In healthy humans, the p53 protein is continually produced and degraded in the cell. The degradation of the p53 protein is, as mentioned, associated with mdm2 binding. In a negative feedback loop mdm2 is itself induced by the p53 protein. However mutant p53 proteins often don't induce mdm2, and are thus able to accumulate at very high concentrations. Worse, mutant p53 protein itself can inhibit normal p53 protein levels.

### DESCRIPTION OF THE INVENTION

It has now been found that alkanoyl L-carnitines are useful agents for increasing the pharmacological activity of chemotherapeutic agents for the treatment or prevention of proliferative diseases or diseases that may be associated with or triggered by persistent angiogenesis, particularly neoplasms, in a mammal, particularly a human.

It is therefore an object of the present invention an alkanoyl L-carnitine or a pharmaceutically acceptable salt thereof, for use as enhancer of the activity of chemotherapeutic agents.

It is a further object of the present invention an alkanoyl L-carnitine or a pharmaceutically acceptable salt thereof, for use as enhancer of the uptake of chemotherapeutic agents by the tumor cells.

It is a further object of the present invention the use of an alkanoyl L-carnitine, or a pharmaceutically acceptable salt thereof, in combination with one or more chemotherapeutic agent; for the preparation of a medicament for the inhibition (delay) of the progression of tumor and/or the treatment of tumor;

in which the dose of alkanoyl L-carnitine to be administered in adult human is higher than 0.5 g/day, preferably higher than 0.8 g/day; most preferably higher than 1 g/day. The pediatric dose may be subject to a reduction of one half or more. This means that for administration to a pediatric patient the dose would typically be higher than 0.250 g/day, preferably higher than 0.4 g/day; most preferably higher than 0.5 g/day.

According to a preferred embodiment of the invention the dose of chemotherapeutic agent to be administered to humans is decreased of from 20% to 30% with respect to the dose recommended for the administration of the same chemotherapeutic agent alone.

Therefore one of the main advantages of the present invention is that the dose of the chemotherapeutic agent (endowed with severe dose-limiting adverse effects) is decreased, when this is administered together with an alkanoyl L-carntine, which is a much more harmless compound, while keeping the sought therapeutic effects.

The administration of alkanoyl L-carnitine is preferably by oral route. The duration of the treatment with alkanoyl L-carnitine may vary from 4 weeks to 12, 24, 32, 48 weeks or even chronic. Preferably the administration is a prolonged administration, i.e. for a period longer than 4 weeks.

According to a preferred embodiment of the invention, the neoplasm to be treated is characterized in that the tumor cells have the wild-type (not mutated) p53 gene.

According to the present invention the alkanoyl L-carnitine is selected from the group consisting of: acetyl, propionyl, valeryl, isovaleryl and butirryl L-carnitine, or a pharmaceutically acceptable salt thereof. Acetyl L-carnitine is preferred.

What is meant by pharmaceutically acceptable salt of alkanoyl L-carnitine is any salt of the latter with an acid that does not give rise to toxic or side effects.

Non-limiting examples of such salts are: chloride, bromide, orotate, aspartate, acid aspartate, acid citrate, magnesium citrate, phosphate, acid phosphate, fumarate and acid fumarate, magnesium fumarate, lactate, maleate and acid maleate, oxalate, acid oxalate, pamoate, acid pamoate, sulphate, acid sulphate, glucose phosphate, tartrate and acid tartrate, glycerophosphate, mucate, magnesium tartrate, 2-amino-ethanesulphonate, magnesium 2-amino-ethane sulphonate, methane sulphonate, choline tartrate, trichloroacetate, and trifluoroacetate.

A list of FDA-approved pharmaceutically acceptable salts is given in the publication Int. J. of Pharm. 33 (1986), 201-217.

According to the present invention the chemotherapeutic agent is selected from the group consisting of: microtubule active agent; a camptothecin derivative; an alkylating agent; an anti-neoplastic anti-metabolite; a platin compound; a topoisomerase inhibitor; a VEGF inhibitor; a tyrosine kinase inhibitor; an EGFR kinase inhibitor; an mTOR kinase inhibitor; an insulin-like growth factor I inhibitor; a Raf kinase inhibitor; a monoclonal antibody; a proteasome inhibitor; a HDAC inhibitor; toxins; imides; paclitaxel; docetaxel; vincristine; vinorelbine; paclitaxel; PS341; R11577; bortezomib; thalidomide; LY355703; bleomicin; epothilone B; temozolamide; 5-FU; gemcitabine; oxaliplatin; cisplatinum; carboplatin; doxorubicin; {6-[4-(4-ethyl-piperazin-1-ylmethyl)-phenyl]-7H-pyrrolo[2,3-d]pyrimidin-4-yl]-((R)-1-phenyl-ethyl)-amine; everolimus; imatinib; erlotinib, bevacizumab, cetuximab, 7-t-butoxyiminomethylcamptothecin and velcade; for simultaneous, concurrent, separate or sequential use in for preventing or treating a proliferative disease.

Any of the combination of components (a) and (b), the method of treating a warmblooded animal comprising administering these two components, a pharmaceutical composition comprising these two components for simultaneous, separate or sequential use, the use of the combination for the delay of progression or the treatment of a proliferative disease or for the manufacture of a pharmaceutical preparation for these purposes or a commercial product comprising such a combination of components (a) and (b), all as mentioned or defined above, will be referred to subsequently also as “combination of the invention” (so that this term refers to each of these embodiments which thus can replace this term where appropriate). Simultaneous administration may, e.g., take place in the form of one fixed combination with two or more active ingredients, or by simultaneously administering two or more active ingredients that are formulated independently. Sequential use (administration) preferably means administration of one (or more) components of a combination at one time point, other components at a different time point, that is, in a chronically staggered manner, preferably such that the combination shows more efficiency than the single compounds administered independently (especially showing synergism). Separate use (administration) preferably means administration of the components of the combination independently of each other at different time points.

Also combinations of two or more of sequential, separate and simultaneous administration are possible, preferably such that the combination component-drugs show a joint therapeutic effect that exceeds the effect found when the combination component-drugs are used independently at time intervals so large that no mutual effect on their therapeutic efficiency can be found, a synergistic effect being especially preferred.

The term “delay of progression”, as used herein, means administration of the combination to patients being in a pre-stage or in an early phase, of the first or subsequent manifestations; or a relapse of the disease to be treated in which patients, e.g., a pre-form of the corresponding disease is diagnosed; or which patients are in a condition, e.g., during a medical treatment or a condition resulting from an accident, under which it is likely that a corresponding disease will develop. “Jointly therapeutically active” or “joint therapeutic effect” means that the compounds may be given separately (in a chronically staggered manner, especially a sequence-specific manner) in such time intervals that they preferably, in the warm-blooded animal, especially human, to be treated, still show a (preferably synergistic) interaction (joint therapeutic effect).

“Pharmaceutically effective” preferably relates to an amount that is therapeutically or in a broader sense also prophylactically effective against the progression of a proliferative disease.

The term “a commercial package” or “a product”, as used herein defines especially a “kit of parts” in the sense that the components (a), which is an alkanoyl L-carnitine derivative and (b), which includes one or more chemotherapeutic agents, as defined above, can be dosed independently or by use of different fixed combinations with distinguished amounts of the components (a) and (b), i.e., simultaneously or at different time points. Moreover, these terms comprise a commercial package comprising (especially combining) as active ingredients components (a) and (b), together with instructions for simultaneous, sequential (chronically staggered, in time-specific sequence, preferentially) or (less preferably) separate use thereof in the delay of progression or treatment of a proliferative disease. The parts of the kit of parts can then, e.g., be administered simultaneously or chronologically staggered, that is at different time points and with equal or different time intervals for any part of the kit of parts. Very preferably, the time intervals are chosen such that the effect on the treated disease in the-combined use of the parts is larger than the effect which would be obtained by use of only any one of the combination partners (a) and (b) as can be determined according to standard methods. The ratio of the total amounts of the combination partner (a) to the combination partner (b) to be administered in the combined preparation can be varied, e.g., in order to cope with the needs of a patient sub-population to be treated or the needs of the single patient which different needs can be due to the particular disease, age, sex, body weight, etc. of the patients. Preferably, there is at least one beneficial effect, e.g., a mutual enhancing of the effect of the combination partners (a) and (b), in particular, a more than additive effect, which hence could be achieved with lower doses of each of the combined drugs, respectively, than tolerable in the case of treatment with the individual drugs only without combination, producing additional advantageous effects, e.g., less side effects or a combined therapeutic effect in a non-effective dosage of one or both of the combination partners (components) (a) and (b), and very preferably a strong synergism of the combination partners (a) and (b).

Both in the case of the use of the combination of components (a) and (b) and of the commercial package, any combination of simultaneous, sequential and separate use is also possible, meaning that the components (a) and (b) may be administered at one time point simultaneously, followed by administration of only one component with lower host toxicity either chronically, e.g., more than 3-4 weeks of daily dosing, at a later time point and subsequently the other component or the combination of both components at a still later time point (in subsequent drug combination treatment courses for an optimal anti-cancer effect) or the like.

The invention further relates to pharmaceutical compositions comprising: (a) an alkanoyl L-carnitine derivative; (b) one or more chemotherapeutic agents; and (c) a pharmaceutically acceptable carrier, if any.

The present invention further relates to a commercial package or product comprising: (a) a pharmaceutical formulation of an alkanoyl L-carnitine derivative; and (b) a pharmaceutical formulation of one or more chemotherapeutic agents for simultaneous, concurrent, separate or sequential use.

The present invention also relates to a method of preventing or treating proliferative diseases in a mammal, particularly a human, with a combination of pharmaceutical agents which comprises:

(a) an alkanoyl L-carnitine selected from the group consisting of acetyl, propionyl, valeryl, isovaleryl and butirryl L-carnitine or a pharmaceutically acceptable salt thereof; and

(b) one or more chemotherapeutic agents.

The present invention further relates to a commercial package or product comprising:

(a) a pharmaceutical formulation of an alkanoyl L-carnitine derivative; and (b) a pharmaceutical formulation of one or more chemotherapeutic agents for simultaneous, concurrent, separate or sequential use.

The combination partners (a) and (b) can be administered together, one after the other or separately in one combined unit dosage form or in two separate unit dosage forms. The unit dosage form may also be a fixed combination.

The Chemotherapeutic Agents

The term “chemotherapeutic agents” is a broad one covering many antineoplastic drugs (used to treat neoplasms) having different mechanisms of action.

According to the present invention combinations of some of these chemotherapeutic agents with an alkanoyl L-carnitine results in improvements in the prevention and treatment of proliferative diseases or diseases that may be associated with or triggered by persistent agiogenesis, such as neoplasms.

Generally, chemotherapeutic agents are classified according to the mechanism of action. Many of the available agents are anti-metabolites of development pathways of various cancers, or react with the DNA of the cancer cells.

The term “chemotherapeutic agent” includes, but is not limited to one or more of the following: a microtubule active agent; an alkylating agent; a camptothecin derivative; an anti-neoplastic anti-metabolite; a platin compound; topoisomerase inhibitor; a compound targeting/decreasing a protein or lipid kinase activity or a protein or lipid phosphatase activity; monoclonal antibodies; proteasome inhibitors; streptomycines; anthraciclines; thiazoles; imides; toxins; and HDAC inhibitors.

The term “microtubule active agent”, as used herein, relates to microtubule stabilizing, microtubule destabilizing agents and microtublin polymerization inhibitors including, but not limited to, taxanes, e.g., paciltaxel and docetaxel; vinca alkaloids, e.g., vinblastine, especially vinblastine sulfate; vincristine, especially vincristine sulfate and vinorelbine; discodermolides; cochicine and epothilonesand derivatives thereof, e.g., epothilone B or a derivative thereof. Paclitaxel is marketed as TAXOL; docetaxel as taxotere; vinblastine sulfate as vinblastin R.P; and vincristine sulfate as farmistin. Also included are the generic forms of paclitaxel, as well as various dosage forms of paclitaxel. Generic forms of paclitaxel include, but are not limited to, betaxolol hydrochloride. Various dosage forms of paclitaxel include, but are not limited to albumin nanoparticle paclitaxel marketed as abraxane; onxol, cytotax. Discodermolide can be obtained, e.g., as disclosed in U.S. Pat. No. 5,010,099. Also included are Epotholine derivatives which are disclosed in U.S. Pat. No. 6,194,181, WO 98/10121, WO 98/25929, WO 98/08849, WO 99/43653, WO 98/22461 and WO 00/31247.

The term “alkylating agent”, as used herein, includes, but is not limited to, cyclophosphamide, ifosfamide, melphalan or nitrosourea (BCNU or Gliadel), or temozolamide (temodar). Cyclophosphamide can be administered, e.g., in the form as it is marketed, e.g., under the trademark cyclostin; and ifosfamide as holoxan.

The term “topoisomerase inhibitors” refers to agents designed to interfere with the action of topoisomerase enzymes (topoisomerase I and II), which are enzymes that control the changes in DNA structure by catalyzing the breaking and rejoining of the phosphodiester backbone of DNA strands during the normal cell cycle. In recent years, topoisomerases have become popular targets for cancer chemotherapy treatments. It is thought that topoisomerase inhibitors block the ligation step of the cell cycle, generating single and double stranded breaks that harm the integrity of the genome. Introduction of these breaks subsequently lead to apoptosis and cell death. The term “topoisomerase inhibitors”, as used herein, includes:
topoisomerase I inhibitors: irinotecan, topotecan, camptothecin, lamellarin D all target type IA topoisomerases and other camptothecin derivatives, such as gimatecan and namitecan.
topoisomerase II inhibitors: etoposide, doxorubicin.

The term camptothecin derivatives as used herein, includes those disclosed in U.S. Pat. No. 6,242,457, incorporated herein by reference.

The term “topoisomerase II inhibitor”, as used herein, includes, but is not limited to, the anthracyclines, such as doxorubicin, including liposomal formulation, e.g., caelyx; daunorubicin, including liposomal formulation, e.g., daunosome; epirubicin; idarubicin and nemorubicin; the anthraquinones mitoxantrone and losoxantrone; and the podophillotoxines etoposide and teniposide. Etoposide is marketed as etopophos; teniposide as vm 26-bristol; doxorubicin as adriblastin or adriamycin; epirubicin as farmorubicin; idarubicin as zavedos; and mitoxantrone as novantron.

The term “anti-neoplastic anti-metabolite” includes, but is not limited to, the protease inhibitor PS341; pirimidine derivatives, 5-fluorouracil (5-FU); capecitabine; gemcitabine; DNA de-methylating agents, such as 5-azacytidine and decitabine; methotrexate; edatrexate; and folic acid antagonists, such as, but not limited to, pemetrexed. Capecitabine can be administered, e.g., in the form as it is marketed, e.g., under the trademark xeloda; and gemcitabine as gemzar.

The term “platin compound”, as used herein, includes, but is not limited to, carboplatin, cisplatin, cisplatinum, oxaliplatin, satraplatin and platinum agents, such as ZD0473. Carboplatin can be administered, e.g., in the form as it is marketed, e.g., carboplat; and oxaliplatin as eloxatin. The term “compounds targeting/decreasing a protein or lipid kinase activity; enzyme inhibitor; or a protein or lipid phosphatase activity; or further anti-angiogenic compounds”, as used herein, includes, but is not limited to, protein tyrosine kinase and/or serine and/or theroine kinase inhibitors or lipid kinase inhibitors, e.g.:

compounds targeting, decreasing or inhibiting the activity of the vascular endothelial growth factor (VEGF) receptors, such as compounds which target, decrease or inhibit the activity of VEGF, especially compounds which inhibit the VEGF receptor, such as, but not limited to, 7/−/−pyrrolo[2,3-d]pyrimidine derivative; BAY 43-9006; isolcholine compounds disclosed in WO 00/09495, such as (4-tert-butyl-phenyl)-94-pyridin-4-ylmethyl-isoquinolin-1-yl)-amine;

compounds targeting, decreasing or inhibiting the activity of the platelet-derived growth factor (PDGF) receptors, such as compounds which target, decrease or inhibit the activity of PDGF receptors, especially compounds which inhibit the PDGF receptor, e.g., a /V-phenyl-2-pyrimidine-amine derivative, e.g., imatinib, SU101, SU6668 and GFB-111;

compounds targeting, decreasing or inhibiting the activity of the fibroblast growth factor (FGF) receptors;

compounds targeting, decreasing or inhibiting the activity of the insulin-like growth factor receptor 1 (IGF-1 R), such as compounds which target, decrease or inhibit the activity of IGF-IR, especially compounds which inhibit the IGF-1 R receptor. Compounds include, but are not limited to, the compounds disclosed in WO 02/092599 and derivatives thereof of 4-amino-5-phenyl-7-cyclobutyl-pyrrolo{2,3-[phi]yrimidine derivatives;

compounds targeting, decreasing or inhibiting the activity of the Trk receptor tyrosine kinase family;

compounds targeting, decreasing or inhibiting the activity of the AxI receptor tyrosine kinase family;

compounds targeting, decreasing or inhibiting the activity of the-c-Met receptor;

compounds targeting, decreasing or inhibiting the activity of the Ret receptor tyrosine kinase;

compounds targeting, decreasing or inhibiting the activity of the Kit/SCFR receptor tyrosine kinase;

compounds targeting, decreasing or inhibiting the activity of the C-kit receptor tyrosine kinases (part of the PDGFR family), such as compounds which target, decrease or inhibit the activity of the c-Kit receptor tyrosine kinase family, especially compounds which inhibit the c-Kit receptor, e.g., imatinib;

compounds targeting, decreasing or inhibiting the activity of members of the c-AbI family and their gene-fusion products, e.g., BCR-AbI kinase, such as compounds which target decrease or inhibit the activity of c-Abl family members and their gene fusion products, e.g., a /V-phenyl-2-pyrimidine-amine derivative, e.g., imatinib, PD180970, AG957, NSC 680410 or PD173955 from ParkeDavis; or BMS354825;

enzyme inhibitor such as imatinib, or the Farnesyl transferase inhibitor R11577;

compounds targeting, decreasing or inhibiting the activity of members of the protein kinase C(PKC) and Raf family of serine/threonine kinases, members of the MEK, SRC, JAK, FAK, PDK and Ras/MAPK family members, or P1(3) kinase family, or of the P1(3)-kinase-related kinase family, and/or members of the cyclin-dependent kinase family (CDK) and are especially those staurosporine derivatives disclosed in U.S. Pat. No. 5,093,330, e.g., midostaurin; examples of further compounds include, e.g., UCN-01; safingol; BAY 43-9006; Bryostatin 1; Perifosine; llmofosine; RO 318220 and RO 320432; GO 6976; lsis 3521; LY333531/LY379196; isochinoline compounds, such as those disclosed in WO 00/09495; FTIs; PD184352 or OAN697, a P13K inhibitor;

compounds targeting, decreasing or inhibiting the activity of protein-tyrosine kinase, such as imatinib mesylate (GLEEVEC); tyrphostin or pyrymidylaminobenzamide and derivatives thereof. A tyrphostin is preferably a low molecular weight (Mr<1500) compound, or a pharmaceutically acceptable salt thereof, especially a compound selected from the benzylidenemalonitrile class or the S-arylbenzenemalonirile or bisubstrate quinoline class of compounds, more especially any compound selected from the group consisting of Tyrphostin A23/RG-50810, AG 99, Tyrphostin AG 213, Tyrphostin AG 1748, Tyrphostin AG 490, Tyrphostin B44, Tyrphostin B44 (+) enantiomer, Tyrphostin AG 555, AG 494, Tyrphostin AG 556; AG957; and adaphostin (4-{[(2,5-dihydroxyphenyl)methyl]amino}-benzoic acid adamantyl ester; NSC 680410, adaphostin);

compounds targeting, decreasing or inhibiting the activity of the epidermal growth factor family of receptor tyrosine kinases (EGFR, ErbB2, ErbB3, ErbB4 as homo- or heterodimers), such as compounds which target, decrease or inhibit the activity of the epidermal growth factor receptor family are especially compounds, proteins or antibodies which inhibit members of the EGF receptor tyrosine kinase family, e.g., EGF receptor, ErbB2, ErbB3 and ErbB4 or bind to EGF or EGF-related ligands, and are in particular those compounds, proteins or monoclonal antibodies generically and specifically disclosed in WO 97/02266, e.g., the compound of Example 39, or in EP 0 564409, WO 99/03854, EP 0520722, EP 0 566226, EP 0 787 722, EP 0 837 063, U.S. Pat. No. 5,747,498, WO 98/10767, WO 97/30034, WO 97/49688, WO 97/38983 and, especially, WO 96/30347, e.g., compound known as CP 358774, WO 96/33980, e.g., compound ZD 1839; and WO 95/03283, e.g., compound ZM105180, e.g., trastuzumab (HERCEPTIN), cetuximab, Iressa, OSI-774, CI-1033, EKB-569, GW-2016, E1.1, E2.4, E2.5, E6.2, E6.4, E2.11, E6.3 or E7.6.3, and {6-[4-(4-ethyl-piperazin-1-ylmethyl)-phenyl]-7H-pyrrolo[2,3-d]pyrimidin-4-yl]-((R)-1-phenyl-ethyl)-amine, erlotinib and gefitinib. Erlotinib can be administered in the form as it is marketed, e.g., TARCEVA, and gefitinib as IRESSA, human monoclonal antibodies against the epidermal growth factor receptor including ABX-EGFR; and

compounds which target, decrease or inhibit the activity/function of serine/theronine mTOR kinase are especially compounds, proteins or antibodies which target/inhibit members of the mTOR kinase family, e.g., RAD, RAD001, CCI-779, ABT578, SAR543, rapamycin and derivatives/analogs thereof, AP23573 and AP23841 from Ariad, everolimus (certican) and sirolimus. Certican (everolimus, RAD) an investigational novel proliferation signal inhibitor that prevents proliferation of T-cells and vascular smooth muscle cells.

The term “monoclonal antibodies”, as used herein, includes, but is not limited to bevacizumab, cetuximab, trastuzumab, lbritumomab tiuxetan, and tositumomab. Bevacizumab can be administered in the form as it is marketed, e.g., AVASTIN; cetuximab as ERBITUX; trastuzumab as HERCEPTIN; rituximab as MABTHERA; ibritumomab tiuxetan as ZEVULIN; and tositumomab as BEXXAR.

The term “proteasome inhibitors”, as used herein, includes compounds which target, decrease or inhibit the activity of the proteosome. Compounds which target, -decrease or inhibit the activity of the proteosome include, but are not limited to, PS-341; MLN 341, bortezomib or velcade.

The term “imides”, as used herein, includes, thalidomide.

The term “toxins” as used herein, includes the cryptomycin analogue LY355703.

The term “HDAC inhibitor”, as used herein, relates to compounds which inhibit the histone deacetylase and which possess anti-proliferative activity. This includes but is not limited to imatinib, the Farnesyl Transferase inhibitor R11577; or compounds disclosed in WO 02/22577, especially [Lambda]/-hydroxy-3-[4-[[(2-hydroxyethyl)[2-(1H-indol-3-yl)ethyl]-amino]methyl]phenyl]-2E-2-propenamide; and [Lambda]/-hydroxy-3-[4-[[{2-(2-methyl-1 W-indol-3-yl)-ethyl]-amino]methyl]phenyl]-2E-2-propenamide; and pharmaceutically acceptable salts thereof. It further especially includes suberoylanilide hydroxamic acid (SAHA); [4-(2-amino-phenylcarbamoyl)-benzyl]-carbamic acid pyridine-3-ylmethyl ester and derivatives thereof; butyric acid, pyroxamide, trichostatin A, oxamflatin, apicidin, depsipeptide, depudecin and trapoxin.

The term “streptomycines”, as used herein, relates to antibiotic drugs used as chemotherapeutic agents, such as bleomicin.

In each case where citations of patent applications or scientific publications are given, in particular with regard to the respective compound claims and the final products of the working examples therein, the subject matter of the final products, the pharmaceutical preparations and the claims is hereby incorporated into the present application by reference to these publications. Comprised are likewise the corresponding derivatives, stereoisomers, pharmaceutically acceptable salts, pharmaceutically acceptable prodrug and esters thereof, as well as the corresponding crystal modifications, e.g., solvates and polymorphs, which are disclosed therein.

The compounds used as active ingredients in the combinations disclosed herein can be prepared and administered as described in the cited documents, respectively.

The structure of the active agents identified by code numbers, generic or trade names may be taken from the actual edition of the standard compendium “The Merck Index” or from databases, e.g., Patents International, e.g., IMS World Publications, or the publications mentioned above and below. The corresponding content thereof is hereby incorporated by reference.

It will be understood that references to the components (a) and (b) are meant to also include the pharmaceutically acceptable salts of any of the active substances. If active substances comprised by components (a) and/or (b) have, e.g., at least one basic center, they can form acid addition salts. Corresponding acid addition salts can also be formed having, if desired, an additionally present basic center. Active substances having an acid group, e.g., COOH, can form salts with bases. The active substances comprised in components (a) and/or (b) or a pharmaceutically acceptable salts thereof may also be used in form of a hydrate or include other solvents used for crystallization. Acetyl L-carnitine is the most preferred combination partner (a).

Carboplatin is an chemotherapeutic agent used against some forms of cancer (mainly ovarian carcinoma, lung, head and neck cancers). It has gained popularity in clinical treatment due to its vastly reduced side-effects compared to its parent compound Cisplatin.

Cisplatin, is a chemotherapeutic agent used to treat various types of cancers, including sarcomas, some carcinomas, lymphomas and germ cell cancers. It was the first member of its class, which now also includes carboplatin and oxaliplatin. Platinum complexes are formed in cells, which bind and cause cross-linking of DNA, ultimately triggering apoptosis, or programmed cell death.

Oxaliplatin is a platinum-based chemotherapy agent in the same family as cisplatin and carboplatin. It is typically administered in combination with fluorouracil and leucovorin for the treatment of colorectal cancer. Compared to cisplatin the two amine groups are replaced by cyclohexyldiamine for improved chemotherapeutic activity.

Bleomycin is a glycopeptide antibiotic used as an anticancer agent. The chemotherapeutical forms used are primarily bleomycin A₂ and B₂. The agent is used in the treatment of Hodgkin lymphoma, squamous cell carcinomas, and testicular cancer, pleurodesis as well as plantar warts.

Vincristine, is a vinca alkaloid from the Madagascar periwinkle. It is a mitotic inhibitor, and is used in cancer chemotherapy. Its main uses are in Hodgkin's lymphoma, acute lymphoblastic leukaemia, and in treatment for nephroblastoma. Like any other vinca alkaloid affects all rapidly dividing cell types including cancer cells, but also intestinal epithelium and bone marrow. The main side-effects of vincristine are peripheral neuropathy, hyponatremia, constipation and hair loss.

Vinorelbine is a semi-synthetic vinca alkaloid agent that is given as a treatment for some types of cancer, including breast cancer and non-small cell lung cancer. Vinorelbine has a number of side-effects that can limit its use: lowered resistance to infection, bruising or bleeding, anaemia, constipation, diarrhoea, nausea, peripheral neuropathy, asthenia, phlebitis.

Epothilone belongs to a new class of cytotoxic molecules identified as potential chemotherapeutic agents.

5-Fluorouracil (5-FU) is a pyrimidine analogue, belonging to the family of agents called antimetabolites. It acts in several ways, but principally as a thymidylate synthesis inhibitor. Like many anti-cancer agents, 5-FU's effects are felt system wide but fall most heavily upon rapidly dividing cells that make heavy use of their nucleotide synthesis machinery, such as cancer cells. Some of its principal use is in colorectal cancer and pancreatic cancer.

The farnesyl transferase inhibitors are a class of experimental chemotherapeutic agents that target protein farnesyl transferase with the downstream effect of preventing the proper functioning of the proteins, which is commonly abnormally active in cancer.

Thalidomide is an oral immunomodulatory agent originally developed as a treatment for insomnia and morning sickness in the 1950s. The mechanism of action of thalidomide is not completely understood. Thalidomide appears to have multiple actions, including the ability to inhibit the growth and survival of myeloma cells in various ways and to inhibit the angiogenesis (Micromedex, Inc.; 2002). Recent Clinical Practice Guidelines for Multiple Myeloma developed by the National Comprehensive Cancer Network (NCCN®, 2004) indicate that the use of thalidomide is an appropriate option as salvage therapy for relapsed or refractory disease and in combination with dexamethasone as initial therapy in patients with advanced myeloma (Durie-Salmon Stage 11 or III). A regulatory application for thalidomide is currently under review by the Food and Agent Administration (FDA) to confirm its efficacy and safety for use in myeloma. Thalidomide is approved in the US for the treatment of the cutaneous manifestations of moderate to severe erythema nodosum leprosum. In addition to myeloma (Br. J. Haematol. 2003; 120:18-26), thalidomide is being evaluated in clinical trials as a treatment for a variety of solid cancers and hematologic malignancies.

The cryptophycin analogue LY355703 is a synthetic product isolated from the blue-green algae, which exerts a potent destabilization of microtubules during mitosis. Many studies were performed to determine the activity of LY355703 in patients with platinum-resistant advanced ovarian cancer and to characterize its toxicity profile. LY355703 has a modest activity in patients with platinum-resistant advanced ovarian cancer. Nevertheless, the considerable rate of disease stabilization in the absence of serious adverse events in this poor-prognosis study population suggests that this novel cryptophycin may deserve further investigation in this setting.

The protease inhibitor PS341 is agent used to treat multiple myeloma that has gotten worse during treatment with other chemotherapeutic agents. It is also used to treat mantle cell lymphoma in patients who have already received at least one other type of treatment. PS-341 is also being studied in the treatment of other types of cancer. It is a type of protease inhibitor and a type of dipeptidyl boronic acid.

The dose of alkanoyl L-carnitine to be used according to the present invention in human is higher than 0.5 g/day, preferably higher than 0.8 g/day; most preferably higher than 1 g/day. The pediatric dose may be subject to a reduction of one half or more. This means that for administration to a pediatric patient the dose would typically be higher than 0.250 g/day, preferably higher than 0.4 g/day; most preferably higher than 0.5 g/day.

In the following are reported the most common therapeutic doses for the antineoplastic agents above mentioned.

5-FU is administered at an appropriate dose in the range from 100-1500 mg daily, e.g., 200-1000 mg/day, such as 200, 400, 500, 600, 800, 900 or 1000 mg/day, administered in one or two doses daily. 5-FU may be administered to a human in a dosage range varying from about 50-1000 mg/m²/day, e.g., 500 mg/m²/day.

DOXORUBICIN may be administered to a human in a dosage range varying from about 10-100 mg/m²/day, e.g., 25 or 75 mg/m²/day, e.g., as single dose.

Epothilone may be administered to a human in a dosage range varying from about 0.1-6 mg/m².

Farnesyl transferase inhibitor may be administered to a human in a dosage range varying from about 100-400 mg/m².

Thalidomide may be administered to a human in a dosage range varying from about 50-500 mg/day.

Cryptomicin analogue LY355703 may be administered to a human in a dosage range varying from about 1-1.5 mg/m².

Protease inhibitor PS341 may be administered to a human in a dosage range varying from about 0.01-10 mg/kg.

Vinorelbine may be administered to a human in a dosage range varying from about 10-50 mg/m².

Vincristine may be administered to a human in a dosage range varying from about 1-2 mg/m².

Bleomicin may be administered to a human in a dosage range varying from about 0.1-1 unit/kg.

Cisplatin may be administered to a human in a dosage range varying from about 30-120 mg/m² about every four weeks.

Carboplatin may be administered to a human in a dosage range varying from about 150-500 mg/m² about every four weeks.

Oxaliplatin may be administered to a human in a dosage range varying from about 50-100 mg/m² every two weeks.

As said before, according to a preferred embodiment of the invention, the dose of chemotherapeutic agent to be administered in combination with an alkanoyl L-carnitine to humans is decreased of from 20% to 30% with respect to the dose recommended for the administration of the same chemotherapeutic agent alone.

Pharmaceutical preparations for the combination therapy for enteral or parenteral administration are, e.g., those in unit dosage forms, such as sugar-coated tablets, capsules or suppositories; and furthermore ampoules. If not indicated otherwise, these formulations are prepared by conventional means, e.g., by means of conventional mixing, granulating, sugar-coating, dissolving or lyophilizing processes. It will be appreciated that the unit content of a combination partner contained in an individual dose of each dosage form need not in itself constitute an effective amount since the necessary effective amount can be reached by administration of a plurality of dosage units. One of skill in the art has the ability to determine appropriate pharmaceutically effective amounts of the combination components.

Preferably, the compounds or the pharmaceutically acceptable salts thereof, are administered as an oral pharmaceutical formulation in the form of a tablet, capsule or syrup; or as parenteral injections if appropriate.

In preparing compositions for oral administration, any pharmaceutically acceptable media may be employed, such as water, glycols, oils, alcohols, flavoring agents, preservatives or coloring agents. Pharmaceutically acceptable carriers include starches, sugars, microcrystalline celluloses, diluents, granulating agents, lubricants, binders and disintegrating agents.

Solutions of the active ingredient, and also suspensions, and especially isotonic aqueous solutions or suspensions, are useful for parenteral administration of the active ingredient, it being possible, e.g., in the case of lyophilized compositions that comprise the active ingredient alone or together with a pharmaceutically acceptable carrier, e.g., mannitol, for such solutions or suspensions to be produced prior to use. The pharmaceutical compositions may be sterilized and/or may comprise excipients, e.g., preservatives, stabilizers, wetting and/or emulsifying agents, solubilizers, salts for regulating the osmotic pressure and/or buffers, and are prepared in a manner known per se, e.g., by means of conventional dissolving or lyophilizing processes. The solutions or suspensions may comprise viscosity-increasing substances, such as sodium carboxymethylcellulose, carboxymethylcellulose, dextran, polyvinylpyrrolidone or gelatin. Suspensions in oil comprise as the oil component the vegetable, synthetic or semisynthetic oils customary for injection purposes.

The isotonic agent may be selected from any of those known in the art, e.g., mannitol, dextrose, glucose and sodium chloride. The infusion formulation may be diluted with the aqueous medium. The amount of aqueous medium employed as a diluent is chosen according to the desired concentration of active ingredient in the infusion solution. Infusion solutions may contain other excipients commonly employed in formulations to be administered intravenously, such as antioxidants.

The present invention further relates to “a combined preparation”, which, as used herein, defines especially a “kit of parts” in the sense that the combination partners (a) and (b) as defined above can be dosed independently or by use of different fixed combinations with distinguished amounts of the combination partners (a) and (b), i.e., simultaneously or at different time points. The parts of the kit of parts can then, e.g., be administered simultaneously or chronologically staggered, that is at different time points and with equal or different time intervals for any part of the kit of parts. The ratio of the total amounts of the combination partner (a) to the combination partner (b) to be administered in the combined preparation can be varied, e.g., in order to cope with the needs of a patient sub-population to be treated or the needs of the single patient based on the severity of any side effects that the patient experiences.

The present invention especially relates to a combined preparation which comprises:

(a) one or more unit dosage forms of an alkanoyl L-carnitine derivative derivative; and

(b) one or more unit dosage forms of an chemotherapeutic agent.

The Diseases to be Treated

The compositions of the present invention are useful for treating proliferative diseases or diseases that are associated with or triggered by persistent angiogenesis, such as neoplasms.

The term “neoplasm” indicates an abnormal mass of tissue as a result of neoplasia. Neoplasia is the abnormal proliferation of cells. The growth of this clone of cells exceeds, and is uncoordinated with, that of the normal tissues around it. It usually causes a tumor. Neoplasms may be benign, pre-malignant or malignant:
benign neoplasms include for example uterine fibroids and melanocytic nevi. They do not transform into cancer.
potentially malignant neoplasms include carcinoma in situ. They do not invade and destroy but, given enough time, will transform into a cancer.
malignant neoplasms are commonly called cancer. They invade and destroy the surrounding tissue, may form metastases and eventually kill the host.

A primary tumor is a tumor growing at the anatomical site, where tumor progression began and proceeded to yield this mass.

Metastasis is the spread of a disease from one organ or part to another non-adjacent organ or part. Only malignant tumor cells and infections have the established capacity to metastasize. Cancer cells can break away, leak, or spill from a primary tumor, enter lymphatic and blood vessels, circulate through the bloodstream, and be deposited within normal tissue elsewhere in the body. Metastasis is one of three hallmarks of malignancy (contrast benign tumors). Most tumors and other neoplasms can metastasize, although in varying degrees (e.g., glioma and basal cell carcinoma rarely metastasize). When tumor cells metastasize, the new tumor is called a secondary or metastatic tumor, and its cells are like those in the original tumor.

According to an embodiment of the present invention the neoplasm to be treated is a primary tumor.

According to a further embodiment of the present invention the neoplasm to be treated is a malignant neoplasm, also called cancer, o a potentially malignant neoplasm.

The combinations of the present invention are particularly useful for treating a cancer which is a breast cancer; lung cancer, including non-small cell lung cancer (NSCLC) and small-cell lung cancer (SCLC); gastrointestinal cancer, including esophageal, gastric, small bowel, large bowel, rectal and colon cancer; glioma, including glioblastoma; sarcoma, such as those involving bone, cartilage, soft tissue, muscle, blood and lymph vessels; ovarian cancer; myeloma; female cervical-cancer; endometrial cancer; head and neck cancer; mesothelioma; renal-cancer; uteran; bladder and urethral cancers; leukemia; lymphoma, prostate cancer; skin cancers; and melanoma. In particular, the inventive compositions are particularly useful for treating: i. a breast cancer; a lung cancer, e.g., non-small cell lung cancer, including non-small cell lung cancer (NSCLC) and small-cell lung cancer (SCLC); a gastrointestinal cancer, e.g., a colorectal cancer; or a genitourinary cancer, e.g., a prostate cancer; ovarian cancer; glioma, including glioblastoma; ii. a proliferative disease that is refractory to the treatment with other chemotherapeutics; or iii. a cancer that is refractory to treatment with other chemotherapeutics due to multidrug resistance.

In a broader sense of the invention, a proliferative disease may furthermore be a hyperproliferative condition, such as a leukemia, lymphoma or multiple myeloma. The combination of the present invention can also be used to prevent or treat diseases that are triggered by persistent angiogenesis, such as Kaposi's sarcoma, leukemia or arthritis.

The present invention also relates to the treatment of pediatric cancers.

An example of pediatric cancer that can be treated or inhibit the progress of the condition according to the present invention are selected from the group consisting of: acute lymphoblastic leukemia, acute myeloid leukemia, adrenocortical carcinoma, astrocytomas, bladder cancer, brain stem glioma, brain stem glioma, central nervous system atypical teratoid/rhabdoid cancer, brain cancer, central nervous system embryonal cancers, brain cancer, astrocytomas, craniopharyngioma, ependymoblastoma, ependymoma, childhood medulloblastoma, medulloepithelioma, pineal parenchymal cancers of intermediate differentiation, supratentorial primitive neuroectodermal cancers and pineoblastoma, breast cancer, bronchial cancers, carcinoid cancer, central nervous system atypical teratoid/rhabdoid cancer, central nervous system embryonal cancers, cervical cancer, chordoma, colorectal cancer, craniopharyngioma, ependymoblastoma, ependymoma, esophageal cancer, extracranial germ cell cancer, gastric cancer, glioma, hepatocellular (liver) cancer, hodgkin lymphoma, kidney cancer, laryngeal cancer, leukemia, acute lymphoblastic/myeloid leukemia, liver cancer, hodgkin lymphoma, non-hodgkin lymphoma, medulloblastoma, medulloepithelioma, mesothelioma, multiple endocrine neoplasia syndrome, acute myeloid leukemia, nasopharyngeal cancer, oral cancer, ovarian cancer, pancreatic cancer, papillomatosis, pineal parenchymal cancers of intermediate differentiation, pineoblastoma and supratentorial primitive neuroectodermal cancers, renal cell cancer, rhabdomyosarcoma, salivary gland cancer, sarcoma, skin cancer, gastric cancer, supratentorial primitive neuroectodermal cancers, thymoma and thymic carcinoma, thyroid cancer and vaginal cancer.

Where a cancer, a cancer disease, a carcinoma or a cancer are mentioned, also metastasis in the original organ or tissue and/or in any other location are implied alternatively or in addition, whatever the location of the cancer and/or metastasis.

The compositions are selectively toxic or more toxic to rapidly proliferating cells than to normal cells, particularly in human cancer cells, e.g., cancerous cancers, the compound has significant anti-proliferative effects and promotes differentiation, e.g., cell cycle arrest and apoptosis.

The pharmaceutical compositions according to the present invention can be prepared by conventional means and are those suitable for enteral, such as oral or rectal, and parenteral administration to mammals including man, comprising a therapeutically effective amount of a camptothecin derivative and at least one chemotherapeutic agent alone or in combination with one or more pharmaceutically acceptable carriers, especially those suitable for enteral or parenteral application.

Pharmaceutical compositions according to the invention may be, e.g., in unit dose form, such as in the form of ampoules, vials, dragees, tablets, infusion bags or capsules.

The effective dosage of each of the combination partners employed in a formulation of the present invention may vary depending on the particular compound or pharmaceutical compositions employed, the mode of administration, the condition being treated and the severity of the condition being treated. A physician, clinician or veterinarian of ordinary skill can readily determine the effective amount of each of the active ingredients necessary to prevent, treat or inhibit the progress of the condition.

For any compound, the therapeutically effective dose can be estimated initially either in cell culture assays, for example, of neoplastic cells, or in animal models, usually mice or rats.

The animal model may also be used to determine the appropriate concentration range and route of administration. Such information can then be used to determine useful doses and routes for administration in humans.

The precise effective dose for a human subject will depend upon the severity of the disease state, general health of the subject, age, weight, and gender of the subject, diet, time and frequency of administration, drug combination(s), reaction sensitivities, and tolerance/response to therapy. This amount can be determined by routine experimentation and is within the judgement of the clinician.

The pharmaceutical composition according to the present invention is composed of active ingredients which are familiar to operators in the medical field and already in use.

Their procurement therefore is very easy, inasmuch as these are products which have been on the market now for a long time and are of a grade suitable for human administration.

The term “therapeutically effective amount” as used herein refers to an amount of a therapeutic agent needed to treat, ameliorate a targeted disease or condition, or to exhibit a detectable therapeutic effect.

For any compound, the therapeutically effective dose can be estimated initially either in cell culture assays, for example, of neoplastic cells, or in animal models, usually mice or rats. The animal model may also be used to determine the appropriate concentration range and route of administration. Such information can then be used to determine useful doses and routes for administration in humans. The precise effective amount for a human subject will depend upon the severity of the disease state, general health of the subject, age, weight, and gender of the subject, diet, time and frequency of administration, drug combination (s), reaction sensitivities, and tolerance/response to therapy. This amount can be determined by routine experimentation and is within the judgement of the clinician. Compositions may be administered individually to a patient or may be administered in combination with other agents, drugs or hormones. The medicament may also contain a pharmaceutically acceptable carrier, for administration of a therapeutic agent. Such carriers include antibodies and other polypeptides, genes and other therapeutic agents such as liposomes, provided that the carrier does not itself induce the production of antibodies harmful to the individual receiving the composition, and which may be administered without undue toxicity. Suitable carriers may be large, slowly metabolised macromolecules such as proteins, polysaccharides, polylactic acids, polyglycolic acids, polymeric amino acids, amino acid copolymers and inactive virus particles.

A thorough discussion of pharmaceutically acceptable carriers is available in Remington's Pharmaceutical Sciences (Mack Pub. Co., N.J. 1991).

Pharmaceutically acceptable carriers in therapeutic compositions may additionally contain liquids such as water, saline, glycerol and ethanol.

Additionally, auxiliary substances, such as wetting or emulsifying agents, pH buffering substances, and the like, may be present in such compositions. Such carriers enable the pharmaceutical compositions to be formulated as tablets, pills, dragees, capsules, liquids, gels, syrups, slurries, suspensions, and the like, for ingestion by the patient. Once formulated, the compositions of the invention can be administered directly to the subject. The subjects to be treated can be animals; in particular human. According to the present invention human pediatric subjects can be treated.

The medicament of this invention may be administered by any number of routes including, but not limited to, oral, intravenous, intramuscular, intra-arterial, intramedullary, intrathecal, intraventricular, transdermal or transcutaneous applications, subcutaneous, intraperitoneal, intranasal, enteral, topical, sublingual, intravaginal, rectal means or locally on the diseased tissue after surgical operation.

Dosage treatment may be a single dose schedule or a multiple dose schedule. The invention will now be illustrated in greater detail by means of non-limiting Examples.

It will be apparent to those skilled in the art, that many modifications, both to materials, and methods, may be practiced with out departing from the purpose and interest of this invention. The examples that follow are not intended to limit the scope of the invention as defined hereinabove or as claimed below.

### Example 1

### Anticancer Effect of Carboplatin in Combination with Acetyl L-Carnitine for the Treatment of NCI—H460 Non-Small Cell Lung Carcinoma

NCI—H460 cancer cells were inoculated subcutaneously (s.c.) in the right flank of CD1 nude mice (3×10⁶/100 μL/mouse). Treatments started three days after cancer injection. Mice were subdivided (8 mice/group) in the following experimental groups: vehicle receiving only sterile water, carboplatin 40 mg/kg, i.p. q4d/wx3w; acetyl L-carnitine (200 mg/kg po, qdx5/wx3w)+carboplatin. Acetyl L-carnitine was administered immediately before the agent given in combination.

To evaluate the anticancer activity, tumor diameters were measured with a Vernier caliper. The formula TV (mm³)=[length (mm)×width (mm)²]/2 was used, where the width and the length are the shortest and the longest diameters of each cancer, respectively. Efficacy of the molecule was evaluated as tumor volume inhibition (TVI %) according to the equation: % TVI=100−[(mean cancer weight of treated mice/mean cancer weight of control group)×100] and Log₁₀ cell kill (LCK) calculated by the formula LCK=(T−C)/3.32×DT, where T and C were the mean times (days) required for treated (T) and control (C) tumors, respectively, to reach 1 cm³, and DT was the doubling time of control tumors. When tumors reached a volume of about 2 cm³, mice were sacrificed by cervical dislocation.

Body weight recording was carried out through the study.

Against NCI—H460 non small cell lung carcinoma xenografted in CD1 nude mice, carboplatin delivered alone at 40 mg/10 ml/kg ip q4d/wx3w was able to reduce the tumor volume of about 48%, but when it was combined with acetyl L-carnitine showed an increase in tumor volume inhibition. TVI was of 79%. No increase in toxicity in the combination group was observed.

The results obtained are reported in the following Table 1.

TABLE 1
Antitumor effect of carboplatin with and without acetyl L-carnitine
against NCI-H460 non-small cell lung carcinoma.

| | DOSE | BWL | LE- | TV ± SE | TVI % | LCK |
|---|---|---|---|---|---|---|
| TREATMENT | (mg/kg) | % | THAL | +31 | +31 | (1 cm³) |
| VEHICLE | 0 | 0 | 0/8 | 1354 ± 344 | / | / |
| CARBO- | 40 ip | 1 | 0/8 | *704 ± 254 | 48 | 0.66 |
| PLATIN | | | | | | |
| ACETYL L- | 200 + 40 | 4 | 0/8 | **290 ± 111 | 79 | 1.50 |
| CARNITINE + | | | | | | |
| CARBO- | | | | | | |
| PLATIN | | | | | | |

Acetyl L-carnitine was given orally (p.o.) according to the schedule qdx5/wx3w (3-7; 10-14; 17-21).
Carboplatin was administered according to the schedule q4d/wx3w at the dose of 40 mg/kg, intra peritoneum (ip.) DT = 3.6 days.
P value was evaluated by Mann-Whitney test (**P < 0.01, *P < 0.05 vs. vehicle treated group).

### Example 2

### Anticancer Effect of Cisplatin in Combination with Acetyl L-Carnitine for the Treatment of NCI—H460 Non-Small Cell Lung Carcinoma

NCI—H460 cancer cells were inoculated subcutaneously (s.c.) in the right flank of CD1 nude mice (3×10⁶/100 μL/mouse). Treatments started three days after tumor injection.

Mice were subdivided (12 mice/group) in the following experimental groups:

1) Vehicle (sterile water) 10 mL/kg, p.o.;
2) cisplatin 4 mg/kg, i.p. q3-4-dx5;
3) acetyl L-carnitine p.o. (200 mg/kg, qdx5/wx4w)+cisplatin;
4) acetyl L-carnitine sub cutaneous s.c. (200 mg/kg, qdx5/wx4w)+cisplatin;
5) acetyl L-carnitine by mini-osmotic pumps s.c. (Alzet, mod 2004) (200 mg/kg/day, qdx28)+cisplatin.

Acetyl L-carnitine was administered immediately before the drug given in combination.

To evaluate the antitumor activity, tumor diameters were measured with a Vernier caliper. The formula TV (mm³)=[length (mm)×width (mm)²]/2 was used, where the width and the length are the shortest and the longest diameters of each tumor, respectively and Log₁₀ cell kill (LCK) calculated by the formula LCK=(T−C)/3.32×DT, where T and C were the mean times (days) required for treated (T) and control (C) tumors, respectively, to reach 1 cm³, and DT was the doubling time of control tumors.

When tumors reached a volume of 1-2 cm³, mice were sacrificed by cervical dislocation. Body weight recording was carried out through the study and mortality was noted.

As shown in Table 2 in all groups which associated cisplatin plus acetyl-L-carnitine an impressive and significative reduction of Tumor volume associated to an increase of LCK were observed compared with cisplatin alone (Table 2).

The results obtained are reported in the following Table 2.

TABLE 2
Antitumor activity of cisplatin in combination with acetyl L-carnitine
in against NCI-H460 NSCLC.

| TREAT- | DOSE | BWL | LE- | | TVI % | LCK |
|---|---|---|---|---|---|---|
| MENT | (mg/kg) | % | THAL. | TV ± SE +31 | +31 | (1 cm³) |
| VEHICLE | 0 | 0 | 0/12 | 1916 ± 303 | / | / |
| CISPLATIN | 6 | 8 | 0/12 | 1138\hat{ } ± 138 | 41 | 0.6 |
| IP | | | | | | |
| ACETYL L- | 200 + 4 | 10 | °1/12 | 407*** ± 90 | 79 | 2.5 |
| CARNITINE | | | (+27) | | (1 n.d.) | |
| P.O. + | | | | | | |
| CISPLATIN | | | | | | |
| ACETYL L- | 200 + 4 | 1 | 0/12 | 710* ± 133 | 63 | 1.3 |
| CARNITINE | | | | | | |
| S.C. + | | | | | | |
| CISPLATIN | | | | | | |
| ACETYL L- | 200 + 4 | 10 | 0/12 | 493** ± 104 | 74 | 2.7 |
| CARNITINE | | | | | (1 n.d.) | |
| O.P. + | | | | | | |
| CISPLATIN | | | | | | |

Acetyl L-carnitine was given p.o. and s.c. according to the schedule qdx5/wx4w (3-7; 10-14; 17-21; 24-28) and by osmotic pumps delivered for 28 days (from day 3 to day 30), 0.25 μL per hour.
Cisplatin was administered according to the schedule q3-4dx5 at the dose of 4 mg/kg 3, 7, 10, 14 and 17 days after the tumor cells injection.
°dead mouse for incorrect oral administration.
DT = 2.6 days.
n.d. = absent tumor lesion.
P value was evaluated by Mann-Whitney test (*P < 0.05, **P < 0.01, ***P < 0.001 vs. cisplatin treated group; \hat{ }P < 0.05 vs vehicle treated group)

### Example 3

Using the experimental condition described in Example 2, the antitumor activity of cisplatin in combination with L-carnitine against NCI—H460 non-small cell lung carcinoma was also evaluated. The results obtained are reported in the following Table 3.

TABLE 3
Antitumor activity of cisplatin in combination with L-carnitine
against NCI-H460 non-small cell lung carcinoma

| | Dose | BWL % | | TV ± SE + | TVI % ± |
|---|---|---|---|---|---|
| Treatment | (mg/kg)/route | max | Leth. | 32 | SE + 32 |
| Vehicle | 0 | 0 | 0/8 | 1748 ± 273 | / |
| Cisplatin | 4/ip | 8 | 0/9 | 345 ± 90 | 80 ± 21 |
| L-carnitine + | 200/po + 4/ip | 12 | 0/9 | 517 ± 68 | 70 ± 9 |
| cisplatin | | | | | |

Tumor cells were inoculated at day 0. Treatment started on day +3 according to the schedule qdx5/wx3w for L-carnitine and q4d/wx3w for cisplatin.
DT = 3.8 days.

The results reported in Table 3 shown that L-carnitine was not able to potentiate the cytotoxic activity of cisplatin when given chronically to NCI—H460 non-small cell lung carcinoma.

### Example 4

### Anticancer Effect of Cisplatin in Combination with Acetyl L-Carnitine for the Treatment of A549 Non-Small Cell Lung Carcinoma

A549 cancer cells were inoculated subcutaneously (s.c.) in the right flank of CD1 nude mice (3×10⁶/100 μL/mouse). Treatments started six days after cancer injection. Mice were subdivided (8 mice/group) in the following experimental groups: cisplatin was given intraperitoneally according to the schedule q3-4d/wx3w and acetyl-L-carnitine according to the schedule qdx5/wx4w.

Acetyl L-carnitine was administered immediately before the agent given in combination.

To evaluate the anticancer activity, tumor diameters were measured with a Vernier caliper. The formula TV (mm³)=[length (mm)×width (mm)²]/2 was used, where the width and the length are the shortest and the longest diameters of each cancer, respectively. Efficacy of the molecule was evaluated as tumor volume inhibition (TVI %) according to the equation: % TVI=100−[(mean cancer weight of treated mice/mean cancer weight of control group)×100]. When tumors reached a volume of about 1 cm3, mice were sacrificed by cervical dislocation.

Body weight recording was carried out through the study.

Against A549 non small cell lung carcinoma xenografted in CD1 nude mice, the combination cisplatin-acetylLcarnitine was able to induce an increase of tumor volume inhibition compared with the effect produced by cisplatin alone.

The results obtained are reported in the following Table 4.

TABLE 4
Antitumor activity of acetyl 1-carnitine in combination with cisplatin
against A549 non-small cell lung carcinoma

| | Dose | BWL | | | |
|---|---|---|---|---|---|
| | (mg/kg)/ | % | | | |
| Treatment | route | max | Lethality | TV ± SE | TVI % ± SE |
| Vehicle | 0 | 0 | 0/8 | 411 ± 71 | / |
| Cisplatin | 4/ip | 8 | 0/8 | 208 ± 55 | 49 ± 12 |
| acetyl 1- | 200/po + | 13 | 0/8 | **142 ± 21 | **65 ± 10 |
| carnitine + | 4/ip | | | | |
| cisplatin | | | | | |

Tumor cells were inoculated at day 0. Treatment started on day +6 according to the schedule qdx5/wx4w for acetyl 1-carnitine and q3-4d/wx3w for cisplatin.
DT = 8 days.
**P < 0.01 vs vehicle-treated group (Mann-Whitney test).

### Example 5

### Anticancer Effect of Cisplatin in Combination with Acetyl L-Carnitine for the Treatment of NCI—H1650 Non-Small Cell Lung Carcinoma

NCI—H1650 cancer cells were resuspended in Medium 199/Matrigel (50:50, v/v) and were injected subcutaneously (s.c.) in the right flank of CD1 nude mice (5×10⁶/200 μL/mouse). Treatments started eleven days after cancer injection.

Mice were subdivided (8 mice/group) in the following experimental groups: cisplatin was given intraperitoneally according to the schedule q3-4-d/wx3w and acetyl-L-carnitine according to the schedule qdx5/wx5w.

Acetyl L-carnitine was administered immediately before the agent given in combination.

To evaluate the anticancer activity, tumor diameters were measured with a Vernier caliper. The formula TV (mm³)=[length (mm)×width (mm)²]/2 was used, where the width and the length are the shortest and the longest diameters of each cancer, respectively. Efficacy of the molecule was evaluated as tumor volume inhibition (TVI %) according to the equation: % TVI=100−[(mean cancer weight of treated mice/mean cancer weight of control group)×100]. When tumors reached a volume of about 1 cm3, mice were sacrificed by cervical dislocation.

Body weight recording was carried out through the study.

Against NCI—H1650 non small cell lung carcinoma xenografted in CD1 nude mice, the combination cisplatin-acetylLcarnitine was able to induce an increase of tumor volume inhibition compared with the effect produced by cisplatin alone.

The results obtained are reported in the following Table 5.

TABLE 5
Antitumor activity of acetyl L-carnitine in combination with cisplatin
against NCI-H1650 non-small cell lung carcinoma

| | Dose | BWL | | | |
|---|---|---|---|---|---|
| | (mg/kg)/ | % | | TV ± SE + | TVI % ± |
| Treatment | route | max | Lethality | 41 | SE + 41 |
| Vehicle | 0 | 0 | 0/8 | 268 ± 82 | / |
| Cisplatin | 4/ip | 10 | 0/8 | 206 ± 32 | 23 ± 4 |
| acetyl | 200/po + | 13 | 0/8 | **102 ± 15 | **62 ± 9 |
| 1-carnitine + | 4/ip | | | | |
| cisplatin | | | | | |

Tumor cells were inoculated at day 0. Treatment started on day +11 according to the schedule qdx5/wx5w for acetyl 1-carnitine and q3-4d/wx3w for cisplatin.
DT = 12 days.
**P < 0.01 vs cisplatin-treated group (Mann-Whitney test).

### Example 6

### Anticancer Effect of Doxorubicin in Combination with Acetyl L-Carnitine for the Treatment of A2780/Dx Multidrug-Resistant Ovarian Carcinoma

A2780/Dx cancer cells were injected subcutaneously (s.c.) in the right flank of CD1 nude mice (5×10⁶/100 μL/mouse). Treatments started eleven days after cancer injection.

Mice were subdivided (10 mice/group) in the following experimental groups: doxorubicin was given intravenously according to the schedule q7dx3 and acetyl-L-carnitine according to the schedule qdx5/wx3w.

Acetyl L-carnitine was administered immediately before the agent given in combination.

To evaluate the anticancer activity, tumor diameters were measured with a Vernier caliper. The formula TV (mm³)=[length (mm)×width (mm)²]/2 was used, where the width and the length are the shortest and the longest diameters of each cancer, respectively. Efficacy of the molecule was evaluated as tumor volume inhibition (TVI %) according to the equation: % TVI=100-[(mean cancer weight of treated mice/mean cancer weight of control group)×100]. and Log₁₀ cell kill (LCK) calculated by the formula LCK=(T−C)/3.32×DT, where T and C were the mean times (days) required for treated (T) and control (C) tumors, respectively, to reach 1 cm³, and DT was the doubling time of control tumors. When tumors reached a volume of about 2 cm³, mice were sacrificed by cervical dislocation.

Body weight recording was carried out through the study.

Against A2780/Dx resistant ovarian cancer xenografted in CD1 nude mice, the combination doxorubicin-acetyl-L-carnitine was able to induce an increase of tumor volume inhibition and log cell kill compared with the effect produced by doxorubicin alone.

The results obtained are reported in the following Table 6.

TABLE 6
Antitumor activity of acetyl L-carnitine in combination with
doxorubicin against A2780/Dx multidrug-resistant ovarian
carcinoma

| | Dose | BWL | | | TVI | LCK |
|---|---|---|---|---|---|---|
| | (mg/kg)/ | % | | TV ± SE | % ± SE | 1 |
| Treatment | route | max | Leth. | +24 | +24 | cm³ |
| Vehicle | 0 | 0 | 0/10 | 2645 ± 426 | / | 0.2 |
| Doxorubicin | 6/iv | 0 | 0/10 | 1059 ± 195 | **60 ± 11 | 0.7 |
| acetyl 1- | 200/po + | 5 | 0/10 | 788 ± 156 | **70 ± 14 | 1.3 |
| carnitine + | 6/iv | | | | | |
| doxorubicin | | | | | | |

Tumor cells were inoculated at day 0. Treatment started on day +3 according to the schedule qdx5/wx3w for acetyl 1-carnitine and q7dx3 for doxorubicin.
DT = 2.9 days.
**P < 0.01 vs vehicle-treated group (Mann-Whitney test).

### Example 7

### Anticancer Effect of Cisplatin in Combination with Acetyl L-Carnitine for the Treatment of IGROV-1 Ovarian Carcinoma

IGROV-1 cancer cells were injected subcutaneously (s.c.) in the right flank of CD1 nude mice (10×10⁶/200 μL/mouse). Treatments started three days after cancer injection.

Mice were subdivided (8 mice/group) in the following experimental groups: cisplatin was given intraperitoneally according to the schedule q3-4d/wx3w and acetyl-L-carnitine according to the schedule qdx-4-5/wx5w.

Acetyl L-carnitine was administered immediately before the agent given in combination.

To evaluate the anticancer activity, tumor diameters were measured with a Vernier caliper. The formula TV (mm³)=[length (mm)×width (mm)²]/2 was used, where the width and the length are the shortest and the longest diameters of each cancer, respectively. Efficacy of the molecule was evaluated as tumor volume inhibition (TVI %) according to the equation: % TVI=100-[(mean cancer weight of treated mice/mean cancer weight of control group)×100]. When tumors reached a volume of about 1-2 cm³, mice were sacrificed by cervical dislocation.

Body weight recording was carried out through the study.

Against IGROV-1 sensitive ovarian cancer xenografted in CD1 nude mice, the combination cisplatin-acetyl-L-carnitine was able to induce an increase of tumor volume inhibition compared with the effect produced by cisplatin alone.

The results obtained are reported in the following Table 7.

TABLE 7
Antitumor activity of acetyl L-carnitine in combination with cisplatin
against IGROV-1 ovarian carcinoma

| | Dose | | | | |
|---|---|---|---|---|---|
| | (mg/kg)/ | BWL % | | TV ± SE + | TVI % ± |
| Treatment | route | max | Leth. | 41 | SE + 41 |
| Vehicle | 0 | 0 | 0/8 | 353 ± 26 | / |
| Cisplatin | 4/ip | 20 | 0/8 | 231 ± 55 | 35 ± 8 |
| acetyl 1- | 200/po + | 15 | 0/8 | ***167 ± 15 | ***53 ± 5 |
| carnitine + | 4/ip | | | | |
| cisplatin | | | | | |

Tumor cells were inoculated at day 0. Treatment started on day +3 according to the schedule qdx4-5/wx5w for acetyl 1-carnitine and q3-4d/wx3w for cisplatin.
DT = 11.9 days.
***P < 0.001 vs vehicle-treated group (Mann-Whitney test).

### EXAMPLES 8-10

### Effect of Acetyl L-Carnitine on Antiproliferative Activity of Cisplatin

The anti-proliferative activity of cisplatin was evaluated in the presence or absence of acetyl 1-carnitine on different tumor cells (NCI—H460 and H1650 non-small cell lung carcinoma cells, A2780/Dx multidrug-resistant ovarian tumor cells and the SJSA-1 (with amplification of mdm2) osteosarcoma cells such as pediatric tumor). Moreover, the activity was also evaluated on two prostate tumor cell lines with p53 wild-type (LnCaP) or p53 null (PC3). To this aim, cells were seeded in 96-wells tissue culture plates and treated for different times with various concentrations of cisplatin in the presence or absence of a concentration (10 mM) ACETYL L-CARNITINE in 0.1% FBS. The number of surviving cells was finally determined by the tetrazolium salt (MTT) assay, as described by Hansen M B et al. (Re-examination and further development of a precise and rapid dye method for measuring cell growth/cell kill. J. Immunol. Methods 119: 203-10, 1989). The cytotoxic potency of the molecules was evaluated by the “ALLFIT” computer program and defined as IC₅₀±SD (drug concentration required for 50% inhibition of cell survival). The statistical comparison between the effect of cisplatin alone and of the combination ACETYL L-CARNITINE-cisplatin was calculated as IC₅₀ value and performed by F-test using the ALLFIT program. Moreover the survival cell in percentage for each concentration of cisplatin with and without acetyl L-carnitine was evaluated to show a possible difference of antiproliferative effect of cisplatin alone and in combination with acetyl L-carnitine. In this case the statistical comparison was performed by Mann-Whitney test.

To test the effects of the compounds on cell growth, tumor cells were seeded in 96-well tissue culture plates at approximately 10% confluence and were allowed to attach and recover for at least 24 h. Tumor cells were exposed to compounds for 72 h or 6 days in 0.1% FBS at 37° C., then medium culture was removed and 100 μL/well of medium were added containing 25 μL/well of a solution 5 mg/mL MTT (final 1 mg/mL). Plates were kept at. 37° C. in incubator with 5% CO₂ for 2 h for the formation of blu chrystals. The supernatant was removed and 100 μL/well of lysant medium were added. Plates were kept under stirring for 60 min. The survival cell was determined as optical density by a Multiskan spectrofluorimeter at 570 nm. The results obtained are reported in the following Tables 8-13.

TABLE 8
Antiproliferative activity of cisplatin with and without a fixed
concentration of acetyl L-carnitine (10 mM) on NCI-H460 non-small
cell lung carcinoma cells (72H of exposure).

| | CELL SURVIVAL % ± SE | | |
|---|---|---|---|
| | | Cisplatin + | Cisplatin vs cisplatin + |
| | | acetyl 1- | acetyl 1-carnitine |
| CONCENTRATION | | carnitine | P value |
| CISPLATIN (NM) | Cisplatin | (10 mM) | (Mann-Whitney) |
| 1250 | 24 ± 0.7 | 18 ± 0.2 | <0.05 |
| 625 | 39 ± 1.0 | 30 ± 0.8 | <0.001 |
| 312 | 61 ± 2.0 | 45 ± 1.6 | <0.05 |
| 156 | 82 ± 2.0 | 59 ± 1.5 | <0.001 |
| 78 | 93 ± 4.0 | 58 ± 5.0 | <0.5 |
| 39 | 96 ± 2.4 | 68 ± 3.9 | <0.5 |

IC₅₀ cisplatin = 0.40 ± 0.05 μM;
cisplatin + acetyl 1-carnitine = 0.13 ± 0.02 μM
P = 0.0001 (F-test)

The results reported in Table 8 show that acetyl L-carnitine was able to potentiate the cytotoxic activity of cisplatin (at about the IC₅₀ or at lower doses) when given chronically (≧72 h of exposure) to NCI—H460 non-small cell lung carcinoma, tumor cells cultured in medium containing 0.1% FBS. A dose of 10 mM of acetyl L-carnitine turned out to be necessary to obtain such results, as the dose of 1 mM resulted ineffective in experiments performed with same schedule and serum conditions. Nevertheless, a low serum concentration (0.1%) in the culture medium resulted a pivotal experimental condition, since 10 mM acetyl L-carnitine failed to increase cisplatin anti-proliferative activity on cells treated for 72 h in medium with 10% FBS. The evaluation of antiproliferative activity was carried out by MTT assay.

TABLE 9
Antiproliferative activity of cisplatin with and without a fixed
concentration of acetyl-L-carnitine (10 mM) on NCI-H1650
non-small cell lung carcinoma cells (6 days of exposure)

| | CELL SURVIVAL % ± SE | | |
|---|---|---|---|
| | | | Cisplatin vs |
| | | Cisplatin + | cisplatin + acetyl 1- |
| CONCENTRATION | | acetyl 1- | carnitine |
| CISPLATIN | | carnitine | P value |
| (NM) | Cisplatin | (10 mM) | (Mann-Whitney) |
| 625 | 81 ± 3.0 | 59 ± 2.0 | <0.05 |
| 312 | 92 ± 2.0 | 61 ± 3.0 | <0.05 |
| 156 | 95 ± 1.0 | 67 ± 1.0 | <0.05 |
| 78 | 98 ± 4.0 | 65 ± 2.0 | <0.5 |

IC₅₀ cisplatin = 1.5 ± 0.1 μM
Cisplatin + acetyl L-carnitine = 0.3 ± 0.06 μM
P < 0.0001 (F-test).

The results reported in Table 9 show that acetyl L-carnitine was able to potentiate the cytotoxic activity of cisplatin (at about the IC50 or at lower doses) when given chronically (≧72 h of exposure) to NCI—H1650 non-small cell lung carcinoma, tumor cells cultured in medium containing 0.1% FBS. A dose of 10 mM of acetyl L-carnitine turned out to be necessary to obtain such results, as the dose of 1 mM resulted ineffective in experiments performed with same schedule and serum conditions. Nevertheless, a low serum concentration (0.1%) in the culture medium resulted a pivotal experimental condition, since 10 mM acetyl L-carnitine failed to increase cisplatin anti-proliferative activity on cells treated for 72 h in medium with 10% FBS. The evaluation of antiproliferative activity was carried out by MTT assay.

TABLE 10
Antiproliferative activity of cisplatin with and without a fixed
concentration of acetyl-L-carnitine (10 mM) on A2780/Dx
multidrug-resistant ovarian carcinoma cells (6 days of exposure)

| | CELL SURVIVAL % ± SE | | |
|---|---|---|---|
| | | | Cisplatin vs |
| | | Cisplatin + | cisplatin + acetyl |
| | | acetyl L- | L-carnitine |
| CONCENTRATION | | carnitine | P value |
| CISPLATIN (NM) | Cisplatin | (10 mM) | (Mann-Whitney) |
| 2500 | 31 ± 1.0 | 25 ± 1.0 | <0.05 |
| 1250 | 40 ± 1.0 | 31 ± 2.0 | <0.05 |
| 625 | 62 ± 2.0 | 42 ± 3.0 | <0.05 |
| 156 | 86 ± 2.0 | 57 ± 3.0 | <0.05 |

IC₅₀ cisplatin = 0.73 ± 0.05 μM
Cisplatin + acetyl L-carnitine = 0.20 ± 0.02 μM
P < 0.0001 (F-test).

The results reported in Table 10 show that acetyl L-carnitine was able to potentiate the cytotoxic activity of cisplatin (at about the IC50 or at lower doses) when given chronically (≧72 h of exposure) to A2780/Dx multi-drug resistant ovarian carcinoma, tumor cells cultured in medium containing 0.1% FBS. A dose of 10 mM of acetyl 1-carnitine turned out to be necessary to obtain such results, as the dose of 1 mM resulted ineffective in experiments performed with same schedule and serum conditions. Nevertheless, a low serum concentration (0.1%) in the culture medium resulted a pivotal experimental condition, since 10 mM acetyl L-carnitine failed to increase cisplatin anti-proliferative activity on cells treated for 72 h in medium with 10% FBS. The evaluation of antiproliferative activity was carried out by MTT assay.

TABLE 11
Pediatric tumor. Antiproliferative activity of cisplatin with and
without a fixed concentration of acetyl-L-carnitine (10 mM)
on SJSA-1 (with amplification of mdm2) osteosarcoma cells
(72 h of exposure followed by 72 h of recovery)

| | Cell survival % ± SE | | |
|---|---|---|---|
| | | | Cisplatin vs |
| | | Cisplatin + | cisplatin + acetyl |
| | | acetyl L- | L-carnitine |
| Concentration | | carnitine | P value |
| cisplatin (nM) | Cisplatin | (10 mM) | (Mann-Whitney) |
| 5000 | 35 ± 1.0 | 28 ± 1.0 | <0.05 |
| 2500 | 64 ± 2.0 | 47 ± 2.0 | =0.05 |
| 1250 | 90 ± 2.0 | 64 ± 2.0 | <0.05 |
| 625 | 90 ± 6.0 | 76 ± 1.0 | =0.05 |
| 312 | 100 ± 3.0 | 81 ± 3.0 | <0.05 |

IC₅₀ cisplatin = 3.2 ± 0.2 μM;
Cisplatin + acetyl L-carnitine = 1.9 ± 0.2 μM
P = 0.027 (F-test).

The results reported in Table 11 shown that acetyl L-carnitine was able to potentiate the cytotoxic activity of cisplatin (at about the 1050 or at lower doses) when given chronically (72 h of exposure) to SJSA-1 osteosarcoma cells cultured in medium containing 0.1% FBS. A dose of 10 mM of acetyl 1-carnitine turned out to be necessary to obtain such results, as the dose of 1 mM resulted ineffective in experiments performed with same schedule and serum conditions. Nevertheless, a low serum concentration (0.1%) in the culture medium resulted a pivotal experimental condition, since 10 mM ACETYL L-CARNITINE failed to increase cisplatin anti-proliferative activity on cells treated for 72 h in medium with 10% FBS. The evaluation of antiproliferative activity was carried out by MTT assay.

TABLE 12
Antiproliferative activity of cisplatin with and without a fixed
concentration of acetyl-L-carnitine (10 mM) on PC3 prostate
carcinoma cells (p53 null) (72 h of exposure)

| | CELL SURVIVAL % ± SE | | |
|---|---|---|---|
| | | Cisplatin + | Cisplatin vs cisplatin + |
| CONCENTRATION | | acetyl L- | acetyl L-carnitine |
| CISPLATIN | | carnitine | P value |
| (NM) | Cisplatin | (10 mM) | (Mann-Whitney) |
| 10000 | 26 | 27 | >0.5 |
| 5000 | 52 | 53 | >0.5 |
| 2500 | 76 | 71 | >0.5 |
| 1250 | 89 | 82 | >0.5 |
| 625 | 97 | 86 | >0.5 |

IC₅₀ cisplatin = 4.63 ± 0.3 μM;
Cisplatin + acetyl l-carnitine = 4.63 ± 0.2 μM
P = 1.0 (F-test)

TABLE 13
Antiproliferative activity of cisplatin with and without a fixed
concentration of acetyl-L-carnitine (10 mM) on LnCaP prostate
carcinoma cells (p53 wild-type) (6 days of exposure)

| | CELL SURVIVAL % ± SE | | |
|---|---|---|---|
| | | | Cisplatin vs |
| | | | cisplatin + acetyl |
| | | Cisplatin + acetyl | L-carnitine |
| CONCENTRATION | | L-carnitine | P value |
| CISPLATIN (NM) | Cisplatin | (10 mM) | (Mann-Whitney) |
| 10000 | 51 ± 3 | 40 ± 2 | <0.05 |
| 5000 | 67 ± 2 | 44 ± 2 | <0.05 |
| 2500 | 74 ± 5 | 46 ± 4 | <0.05 |
| 1250 | 92 ± 2 | 52 ± 3 | <0.05 |

IC₅₀ cisplatin = 7.8 ± 1.6 μM;
Cisplatin + acetyl l-carnitine = 1.6 ± 0.5 μM
P = 0.005 (F-test).

The results reported in Tables 12 and 13 show that acetyl L-carnitine was able to potentiate the cytotoxic activity of cisplatin (at about the 1050 or at lower doses) when given chronically (≧72 h of exposure) only on the tumor cell line with p53 wild-type (LnCaP) and not on the tumor cell line with P53 null (PC3), both cultured in medium containing 0.1% FBS. The evaluation of antiproliferative activity was carried out by MTT assay.

### Example 11

### Anticancer Effect of Cisplatin in Combination with Acetyl L-Carnitine for the Treatment of SW620 Colon Carcinoma with Mutant p53

SW620 tumor cells were injected subcutaneously (s.c.) in the right flank of CD1 nude mice (3×10⁶/200 μL/mouse). Treatments started three days after cancer injection.

Mice were subdivided (8 mice/group) in the following experimental groups: cisplatin was given intraperitoneally according to the schedule q4dwx3w and acetyl-L-carnitine according to the schedule qdx5wx3w.

Acetyl L-carnitine was administered immediately before the agent given in combination.

To evaluate the anticancer activity, tumor diameters were measured with a Vernier caliper. The formula TV (mm³)=[length (mm)×width (mm)²]/2 was used, where the width and the length are the shortest and the longest diameters of each cancer, respectively. Efficacy of the molecule was evaluated as tumor volume inhibition (TVI %) according to the equation: % TVI=100−[(mean cancer weight of treated mice/mean cancer weight of control group)×100]. When tumors reached a volume of about 1 cm³, mice were sacrificed by cervical dislocation.

Body weight recording was carried out through the study.

The results obtained are reported in the following Table 14.

TABLE 14
Antitumor activity of acetyl L-carnitine in combination with cisplatin
against SW620 colon carcinoma with mutant p53.

| | Dose | BWL % | | TV ± SE + | TVI % ± |
|---|---|---|---|---|---|
| Treatment | (mg/kg)/route | max | Leth. | 20 | SE + 20 |
| Vehicle | 0 | 0 | 0/8 | 537 ± 59 | / |
| Cisplatin | 4/ip | 11 | 0/8 | 244 ± 45 | 55 ± 10 |
| acetyl L- | 200/po + 4/ip | 6 | 0/8 | 329 ± 42 | 39 ± 5 |
| carnitine + | | | | | |
| Cisplatin | | | | | |

Tumor cells were inoculated at day 0. Treatment started on day +3 according to the schedule qdx5/wx3w for acetyl L-carnitine and q4d/wx3w for cisplatin.
DT = 6.2 days.

The results obtained show that the combination cisplatin+acetyl L-carnitine was not able to induce an increase of tumor volume inhibition compared with the effect produced by cisplatin alone, using the SW620 colon carcinoma with mutant p53.

1. Method of enhancing the activity of one or more chemotherapeutic agent in the prevention or treatment of a proliferative disease or disease associated with or triggered by persistent angiogenesis in a mammal, comprising:
administering to an adult human an alkanoyl L-carnitine, or a pharmaceutically acceptable salt thereof at a dose higher than 05.g/day in combination with a chemotherapeutic agent selected from the group consisting of: an alkylating agent; an anti-neoplastic anti-metabolite; a platin compound; a topoisomerase inhibitor; a VEGF inhibitor; a tyrosine kinase inhibitor; an EGFR kinase inhibitor; an mTOR kinase inhibitor; an insulin-like growth factor I inhibitor; a Raf kinase inhibitor; a monoclonal antibody; a proteasome inhibitor; a HDAC inhibitor; toxins; and imides.

2. Method of enhancing uptake of one or more chemotherapeutic agent by the tumor cells in the prevention or treatment of a proliferative disease or disease associated with or triggered by persistent angiogenesis in a mammal, comprising:
administering an alkanoyl L-carnitine, or a pharmaceutically acceptable salt thereof to an adult human at a dose higher than 0.5 g/day in combination with a chemotherapeutic agent is selected from the group consisting of: vincristine; vinorelbine; PS341; R11577; bortezomib; thalidomide; LY355703; bleomicin; epothilone B; temozolamide; 5-FU; gemcitabine; oxaliplatin; cisplatinum; carboplatin; doxorubicin; {6-[4-(4-ethyl-piperazin-1-ylmethyl)-phenyl]-7H-pyrrolo[2,3-d]pyrimidin-4-yl]-((R)—I-phenyl-ethyl)-amine; everolimus; imatinib; erlotinib, bevacizumab, cetuximab, 7-t-butoxyiminomethylcamptothecin and velcade.

3. Method for the prevention or treatment of a proliferative disease or disease associated with or triggered by persistent angiogenesis in a mammal, comprising:
administering to an adult human a medicament comprising
an alkanoyl L-carnitine, or a pharmaceutically acceptable salt thereof at a dose higher than 0.5 g/day; and
a chemotherapeutic agent is selected from the group consisting of: an alkylating agent; an anti-neoplastic anti-metabolite; a platin compound; a topoisomerase inhibitor; a VEGF inhibitor; a tyrosine kinase inhibitor; an EGFR kinase inhibitor; an mTOR kinase inhibitor; an insulin-like growth factor I inhibitor; a Raf kinase inhibitor; a monoclonal antibody; a proteasome inhibitor; a HDAC inhibitor; a toxin; and an imide.

4. Method of claim 3, wherein
the chemotherapeutic agent is selected from the group consisting of: vincristine; vinorelbine; PS341; R11577; bortezomib; thalidomide; LY355703; bleomicin; epothilone B; temozolamide; 5-FU; gemcitabine; oxaliplatin; cisplatinum; carboplatin; doxorubicin; {6-[4-(4-ethyl-piperazin-1-ylmethyl)-phenyl]-7H-pyrrolo[2,3-d]pyrimidin-4-yl]-((R)—I-phenyl-ethyl)-amine; everolimus; imatinib; erlotinib, bevacizumab, cetuximab, 7-t-butoxyiminomethylcamptothecin and velcade.

5. Method according to claim 3, wherein the alkanoyl L-carnitine is selected from the group consisting of acetyl, propionyl, valeryl, isovaleryl and butirryl L-carnitine, preferably acetyl L-carnitine.

6. Method according to claim 3, wherein the pharmaceutically acceptable salt of the alkanoyl L-carnitine is selected from the group consisting of: chloride, bromide, orotate, aspartate, acid aspartate, acid citrate, magnesium citrate, phosphate, acid phosphate, fumarate and acid fumarate, magnesium fumarate, lactate, maleate and acid maleate, oxalate, acid oxalate, pamoate, acid pamoate, sulphate, acid sulphate, glucose phosphate, tartrate and acid tartrate, glycerophosphate, mucate, magnesium tartrate, 2-amino-ethanesulphonate, magnesium 2-amino-ethanesulphonate, methanesulphonate, choline tartrate, trichloroacetate, and trifluoroacetate.

7. Method according to claim 3, wherein the medicament is for the treatment of a neoplasm.

8. Method according to claim 7, wherein the neoplasm is a malignant neoplasm or a cancer.

9. Method according to claim 7, wherein the neoplasm is a primary tumor.

10. Method according to claim 7, wherein the neoplasm is characterized in that the tumor cells have the wild-type (not mutated) p53 gene.

11. Method according to claim 8, in which the cancer is selected from the group consisting of: non-small cell lung cancer; small-cell lung cancer; gastrointestinal cancer; glioma; sarcoma; ovarian cancer; myeloma; female cervical cancer; endometrial cancer; head and neck cancer; mesothelioma; renal cancer; uteran cancer; bladder and urethral cancers; leukemia; prostate cancer; skin cancers; melanoma; leukemia; lymphoma; and multiple myeloma.

12. Method according to claim 8, in which the cancer is a pediatric cancer.

13. Method according to claim 12, in which the pediatric cancer is selected from the group consisting of: acute lymphoblastic leukemia, acute myeloid leukemia, adrenocortical carcinoma, astrocytomas, bladder cancer, brain stem glioma, central nervous system atypical teratoid/rhabdoid cancer, brain cancer, central nervous system embryonal cancers, brain cancer, astrocytomas, craniopharyngioma, ependymoblastoma, ependymoma, childhood medulloblastoma, medulloepithelioma, pineal parenchymal cancers of intermediate differentiation, supratentorial primitive neuroectodermal cancers and pineoblastoma, breast cancer, bronchial cancers, carcinoid cancer, central nervous system atypical teratoid/rhabdoid cancer, central nervous system embryonal cancers, cervical cancer, chordoma, colorectal cancer, craniopharyngioma, ependymoblastoma, ependymoma, esophageal cancer, extracranial germ cell cancer, gastric cancer, glioma, hepatocellular (liver) cancer, hodgkin lymphoma, kidney cancer, laryngeal cancer, leukemia, acute lymphoblastic/myeloid leukemia, liver cancer, Hodgkin lymphoma, non-Hodgkin lymphoma, medulloblastoma, medulloepithelioma, mesothelioma, multiple endocrine neoplasia syndrome, acute myeloid leukemia, nasopharyngeal cancer, oral cancer, ovarian cancer, pancreatic cancer, papillomatosis, pineal parenchymal cancers of intermediate differentiation, pineoblastoma and supratentorial primitive neuroectodermal cancers, renal cell cancer, rhabdomyosarcoma, salivary gland cancer, sarcoma, skin cancer, gastric cancer, supratentorial primitive neuroectodermal cancers, thymoma and thymic carcinoma, thyroid cancer and vaginal cancer.

14. Method according to claim 12, wherein the alkanoyl L-carnitine, or a pharmaceutically acceptable salt thereof, is for administration to a pediatric patient at a dose higher than 0.250 g/day.

15. Method according to claim 3, wherein the alkanoyl L-carnitine and/or the chemotherapeutic agent are administered via a route selected from: oral, parenteral, intravenous, intramuscular, intra-arterial, intramedullary, intrathecal, intraventricular, transdermal or transcutaneous, subcutaneous, intraperitoneal, intranasal, enteral, topical, sublingual, intravaginal, rectal means or locally on the diseased tissue after surgical operation.

16. Method according to claim 3, wherein the administration of the alkanoyl L-carnitine and the chemotherapeutic agent is simultaneous, sequential or separate as well as in a single dose schedule or in a multiple dose schedule.

17. Method according to claim 3, wherein the dose of chemotherapeutic agent to be administered to humans is decreased of from 20% to 30% with respect to the dose recommended for the administration of the same chemotherapeutic agent alone.

18. Method of claim 1, wherein the dose of the alkanoyl L-carnitine is higher than 0.8 g/day.

19. Method of claim 1, wherein the dose of the alkanoyl L-carnitine is higher than 1 g/day.

20. Method of claim 14, wherein the dose of the alkanoyl L-carnitine is higher than 0.4 g/day.

21. Method of claim 14, wherein the dose of the alkanoyl L-carnitine is higher than 0.5 g/day
